(19) United States
(12) Patent Application Publication
(10) Pub. No.: US 2020/0066384 A1
(43) Pub. Date: Feb. 27, 2020

(54) CLINICAL REPORT WITH AN ACTIONABLE RECOMMENDATION

(71) Applicant: KONINKLIJKE PHILIPS N.V., EINDHOVEN (NL)

(72) Inventors: Amir Mohammad TAHMASEBI MARAGHOOSH, ARLINGTON, MA (US); Merlijn SEVENSTER, HAARLEM (NL); Nicole SCHADEWALDT, NORDERSTEDT (DE); Lucas OLIVEIRA, WILLMINGTON, MA (US)

(21) Appl. No.: 16/607,740

(22) PCT Filed: Apr. 30, 2018
(86) PCT No.: PCT/EP2018/061026

§ 371 Date: Oct. 24, 2019

Related U.S. Application Data
(60) Provisional application No. 62/491,777, filed on Apr. 28, 2017.

(51) Int. Cl. G16H 20/00; G16H 15/00; G16H 40/20; G06F 17/27

(52) U.S. Cl. CPC G16H 20/00; G06F 17/2705; G16H 40/20; G16H 15/00

(57) ABSTRACT

A system (100) includes a parser engine (120), a context inferring engine (130), and an action selection engine (140). The parser engine (120) detects a recommendation in a received clinical report (112) for a patient. The context inferring engine (130) extracts and normalizes recommendation elements in the detected recommendation. The action selection engine (140) generates a patient actionable clinical report (142) based on the received clinical report and the extracted and normalized recommendation elements.

### FIELD OF THE INVENTION

The following generally relates to clinical reports, and more specifically to a clinical report with an actionable recommendation.

### BACKGROUND OF THE INVENTION

A healthcare practitioner, such as a radiologist, reviews an image(s) of a patient from an imaging procedure or study and prepares a clinical report. The prepared clinical report, e.g., formatted as narrative text, includes relevant findings and/or incidental findings. For example, a computed tomography (CT) image from a CT abdomen imaging study of a patient includes the liver and portions of the lung. In this example, the CT imaging study is ordered in response to patient conditions suggesting cirrhosis of the liver. The relevant findings include information confirming the cirrhosis, and the incidental findings include information indicating potential lung nodules. Based on these findings, the healthcare practitioner includes recommendations for follow-ups in the clinical report, such as another patient imaging study, laboratory test, or other medical examination.

The clinical report is sent to a referring healthcare practitioner directing the care of the patient. The referring healthcare practitioner reviews the report. The recommendations included in the clinical report are sometimes not followed, such as when the referring healthcare practitioner fails to act upon the recommendations, the recommendations are not clearly identified or difficult to interpret, the patient fails to schedule an appointment for an examination scheduled in response to a recommendation, the patient misses a scheduled appointment for an examination which was scheduled in response to a recommendation, and the like. Unfortunately, this could lead to impact the safety of the patients, e.g., resulting in missed or delayed diagnoses, and/or increased risk of liability for the clinician and/or healthcare facility.

One approach to mitigate missing a recommendation in a report is for a healthcare provider organization to hire assistants that separately review clinical reports. The hired assistants try to ensure that recommendations are not missed, to schedule appointments between the patient and an appropriate follow-up healthcare practitioner or organization, and to call patients to remind them of scheduled appointments. Properly identifying recommendations for follow-ups in a clinical report involves time, training and medical knowledge. Scheduling appointments and reminders include time and communication methods with the patient and a scheduler of a follow-up appointment. The communication methods include coordination problems, such as matching the patient schedule availability to follow-up schedule openings. The communication methods include delays, such as responses to inquiries or notifications between the healthcare provider and the patient or the follow-up scheduler.

### SUMMARY OF THE INVENTION

Aspects described herein address the above-referenced problems and others.

The following describes embodiments of a system and method for patient actionable clinical reports. A patient portal provides direct access for reading a clinical report which includes a patient actionable recommended action. In some embodiments, the patient portal provides a set of tools relevant to the patient actionable recommended action.

In one aspect, a system includes a parser engine, a context inferring engine, and an action selection engine. The parser engine detects a recommendation in a received clinical report for a patient. The context inferring engine extracts and normalizes recommendation elements in the detected recommendation. The action selection engine generates a patient actionable clinical report based on the received clinical report and the extracted and normalized recommendation elements.

In another aspect, a method includes detecting a recommendation in a received clinical report for a patient. Recommendation elements extracted and normalized in the detected recommendation. A patient actionable clinical report is generated based on the received clinical report and the extracted and normalized recommendation elements.

In another aspect, a non-transitory computer-readable storage medium carrying instructions controls one or more processors to detect a recommendation in a received clinical report for a patient. Recommendation elements extracted and normalized in the detected recommendation. A patient actionable clinical report is generated based on the received clinical report and the extracted and normalized recommendation elements.

These and other aspects of the invention will be apparent from and elucidated with reference to the embodiment(s) described hereinafter.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of an electronic clinical report system.

FIG. 2 illustrates an example of a displayed clinical report with an actionable recommendation.

FIG. 3 flowcharts an embodiment of a method of generating a clinical report with an actionable recommendation.

### DETAILED DESCRIPTION OF EMBODIMENTS

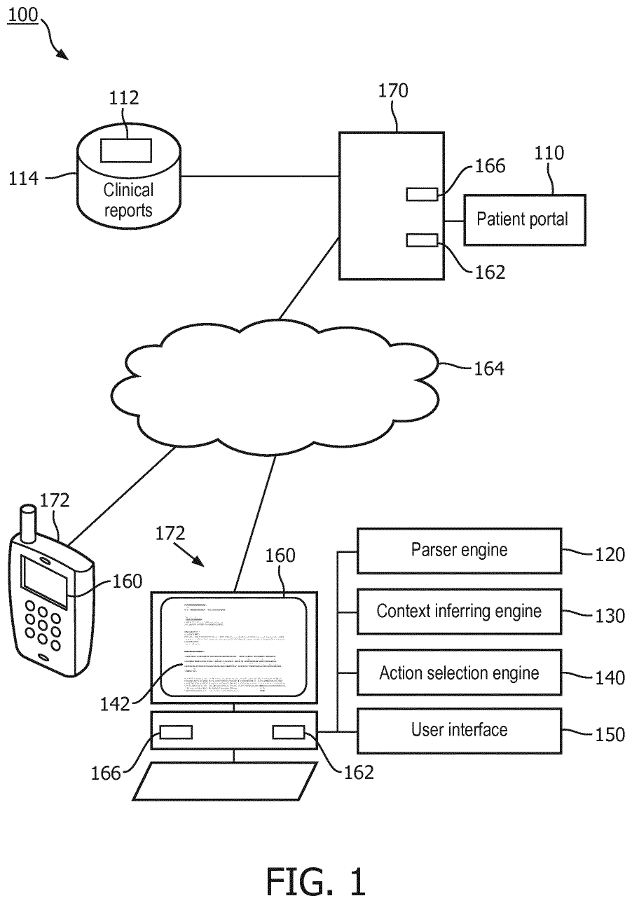

With reference to FIG. 1, an embodiment of a system 100 configured for generating patient actionable clinical reports (e.g., a clinical report with an actionable recommendation) is schematically illustrated. A patient portal 110 retrieves a clinical report 112 from electronic storage 114, such as from a hospital information system (HIS) or database, electronic medical record (EMR), picture archiving and communication system (PACS), radiology information system (RIS), local storage system and the like. For example, radiology clinical reports can be accessed through a PACS application programming interface (API) based on a unique patient identifier such as a hospital-specific medical record number (MRN) only for that patient, or a combination of characteristic information items, such as date of birth, name, phone number, Social Security Number, etc., and/or other information.

A parser engine 120 receives the clinical report 112 and detects a recommendation(s). The parser engine 120 uses natural language processing (NLP) techniques to parse the clinical report 112. The clinical report 112 is parsed into sections, paragraphs and sentences, which can be implemented using pattern matching, such as with a regular expression, statistical matching, such as with a maximum entropy class selection or a word2vec vector space, or using machine learning. The parser engine 120 searches by paragraph for keywords and/or concepts indicative of a recommendation for follow-up. In one non-limiting instance, the search is performed without accounting for common lexical variants and typing mistakes. In another non-limiting instance, the search is performed accounting for common lexical variants and typing mistakes. The parser engine 120 can use headings and/or other structural aspects of the clinical report 112 to locate keywords and/or concepts indicative of a recommendation for follow-up. For example, recommendations can be found in paragraphs under a report heading of “Impression:” rather than a report heading of “Technique:” or other sections of explaining the examination performed.

An inferring context engine 130 extracts and normalizes recommendation elements from a paragraph(s) detected to have a keyword and/or concept indicative of a recommendation for follow-up. The extracted recommendation elements are normalized with respect to machine-interpretable value sets of recommendation elements, such as can be derived from a medical ontology like RadLex®. Recommendation elements include an action and a time frame and can include additional elements according to the action. For example, an action can include an imaging examination, a lab evaluation, and/or a completed questionnaire. Examples of time frame can include 3 months, 6 months, 1 year from the date of the clinical report 112.

The inferring context engine 130 can identify additional elements, such as modality, anatomy, and a reason for the examination in a recommendation for a follow up imaging examination or study. For example, a modality can include computed tomography (CT), magnetic resonance (MR), computed radiography (CR), positron emission tomography (PET), single proton emission computed tomography (SPECT), ultrasound (US), and the like. Examples of anatomy include head, breast, abdomen, or chest. Examples of a reason for an imaging examination include monitor lung nodule, revise emphysema function, etc.

For example, a sentence “If this patient has risk factors for lung cancer, then a CT chest is recommended in 12 months for further evaluation” includes “recommend” as a lexical word base indicative of a recommendation in the associated sentence text. That is, the word “recommend” indicates an action element. “CT Chest” is inferred as an action element indicative of an imaging study. The time frame is inferred as 12 months from the date of the clinical report 112. Thus, the inferring context engine 130 identifies additional elements as a CT modality, a chest anatomy, and monitor lung nodules as a reason for an imaging examination.

An action selection engine 140 identifies a set of tools that are actionable for the patient and associated with the detected recommendation. For example, with the recommended action element of an imaging study and related elements, a tool set includes a tool for locating nearby healthcare providers of the imaging study, a tool for additional information on the imaging study or reason for the imaging study, a tool to chat with a healthcare practitioner about the imaging study or reason for the imaging study, a tool to schedule the imaging study, a tool to communicate via voice with a healthcare practitioner about the imaging study or reason for the imaging study. Non-identified tools for the imaging study may include tools for completing certain questionnaires, scheduling a laboratory test, and the like. The action selection engine 140 generates a patient actionable clinical report 142. The patient actionable clinical report 142 indicates the patient actionable recommendation within the clinical report, such as with an underline, color change, highlight, and the like. In some embodiments, each patient actionable recommendation includes a hyperlink that invokes a corresponding set of tools. The patient actionable recommendation is a detected recommendation in which the patient can complete a task or next step to satisfy, at least in part, the follow-up. For example, the patient schedules a CT chest imaging study in a 12 month timeframe as a next step to satisfy a recommended CT chest imaging study follow-up in 12 months.

A user interface 150 displays the generated patient actionable clinical report 142 on a display device 160. The display can include icons or links for each of the corresponding set of tools. In some embodiments, the user interface 150 can modify the display in response to selecting each indicated patient actionable recommendation.

The patient portal 110, the parser engine 120, the context inferring engine 130, the action selection engine 140, and the user interface 150 are suitably embodied by one or more configured processors 162, such as a digital processor, a microprocessor, an electronic processor, an optical processor, a multi-processor, a distribution of processors including peer-to-peer or cooperatively operating processors, client-server arrangement of processors, and the like, communicatively connected to a network 164 and configured to receive and parse the clinical report 112, detect the recommendations, identify selected tool sets, generate the patient actionable clinical report 142 and operate the display device 160 to display icons or links for each of the corresponding tool set(s).

In some embodiments, the configured processors comprise a web based client server arrangement, which includes a computer server 170, such as a server configured with hypertext transfer protocol (HTTP), and a client computing device 172 configured with a web browser. In some embodiments, the client server arrangement uses non-web based communication, such as with an app on a client device specific to the patient portal 110. The client computing device 172 can include a laptop, desktop, tablet, TV, smartphone, body worn device, and the like. The network can include data networks, cellular networks, public networks, private networks, combinations thereof, and the like.

The configured processors 162 execute at least one computer readable instruction stored in computer readable storage medium 166, such as an optical disk, a magnetic disk, semiconductor memory of a computing device with the configured processor, which excludes transitory medium and includes physical memory and/or other non-transitory medium to perform the disclosed techniques. The configured processor may also execute one or more computer readable instructions carried by a carrier wave, a signal or other transitory medium. The lines between components represented in the diagram represent communications paths.

The stored clinical reports 112 are suitably embodied by a computer storage medium, such as local disk, cloud storage, remote storage, and the like, accessed by one or more configured computer processors 162. The display device 152 is suitably embodied as a computer display, smartphone display, projector, body worn display, and the like.

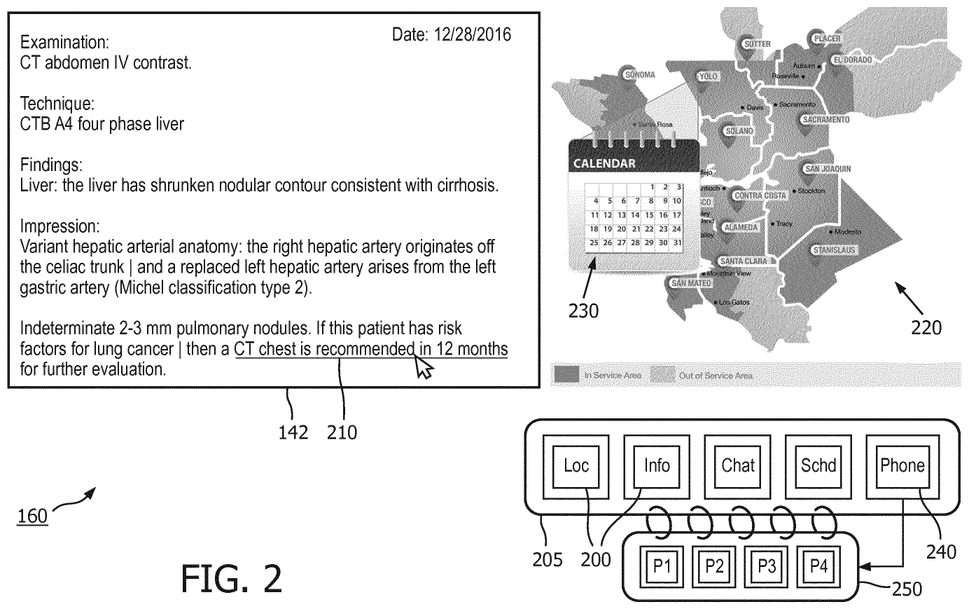

With reference to FIG. 2, an example displayed patient actionable clinical report 142 and icons 200 indicating the corresponding set of tools 205 are illustrated. The patient actionable clinical report 142 includes the indicator 210 of a patient actionable recommendation. The patient actionable recommendation is a “CT chest is recommended in 12 months,” which is underlined and hyperlinked to the displayed set of tools 205. In some instances, the indicator 210 of a patient actionable recommendation emphasizes any required action from the displayed patient actionable clinical report 142. In some instances, the displayed set of tools 205 enables the patient to actively take care of his healthcare management by acting on the recommendation. In some instances, the set of tools 205 reduces the patient effort to follow-up by providing connections and information beyond the stated recommendation and increases the likelihood that the recommendation will be followed.

In some embodiments, the user interface 150 automatically invokes the most likely next action or tool from the set of tools 205. For example, a likely next action or actions are to view the locations of nearby healthcare facilities that provide imaging procedures, such as in a list format or example displayed map format 220 according to a locator or mapping tool. In some embodiments, the nearby locations can use a global positioning system (GPS) location of the computing device 172, a stored address of the patient in the patient portal, addresses of the healthcare facilities providing the recommended elements, and the like as a basis for determining the nearby healthcare providers. The display includes a calendar display 230 set forward according to the time frame element. That is, using the prior stated recommendation example of “CT Chest is recommended in 12 months”, the calendar display 230 of a scheduling tool is set forward to the 12-month time frame.

Patient actionable recommendations can include completing a questionnaire of various factors or input related to for example, depression level, pain level, or quality of life. The icon representing the tool can include a direct link to an appropriate online form. In some embodiments, the tool can include a calendar invite with an estimate of the time to complete the questionnaire. Tools can include links for additional information, such as links to background literature on how to prepare for an imaging study. The links can include contextual elements, such as recommendation elements, which facilitate access to the background literature, such as pre-formatted search terms. Tools can include communication links, such as to contact a referring physician to obtain a referral.

Tools in the set of tools 205 can use additional data stored in the patient portal 110, such as insurance coverage, patient address or other demographics, or other profile information. Tools in the set of tools 205 can use additional information concerning hospital network locations, healthcare communication channels, laboratory and/or examination schedules, and the like. For example, nearby healthcare providers displayed in a locator tool can be indicated as in-network or out-of-network, in service area or out of service area, and the like. The locator tool can include additional information, such as a fee or estimated fee for the examination by each provider location, provider locations using shared access to images, and the like.

In another example, if another provider location, e.g. imaging center, is scheduled that is different from the provider location issuing the clinical report 112, the scheduling tool can include an automated request to transfer patient files, such as patient images from the clinical report 112 issuing provider location to the scheduled provider location, which can allow for comparison of the images. Another example includes scheduling notifications to the referring or managing physician, which can be used for patient tracking.

In another example, an icon 240 representing the tool for phone or voice communication channels to healthcare practitioners includes a submenu of icons 250 representing different types of healthcare practitioners, P1-P4, such as a nurse, physician's assistant, referring physician, radiologist, and the like. Selection of an icon 250 can invoke phone or voice communications with the represented healthcare practitioner.

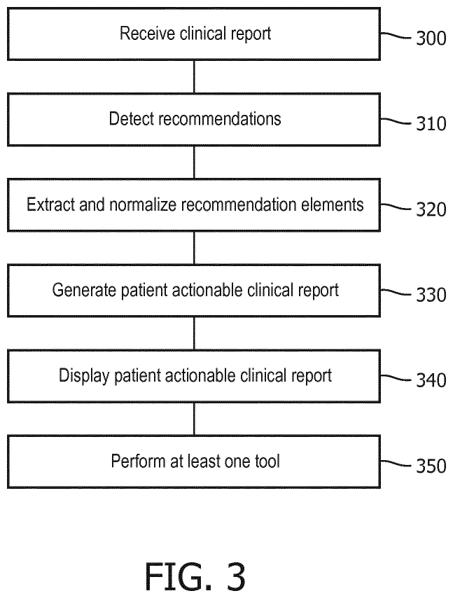

With reference to FIG. 3, an embodiment of a method of generating a patient actionable clinical report 142 and corresponding set of tools 205 is flowcharted.

At 300, a clinical report 112 is received.

At 310, a recommendation(s) is detected in the clinical report 112.

At 320, elements from the detected recommendations are extracted and normalized. The recommendation elements include the action and the timeframe and can include additional elements according to the action.

At 330, the patient actionable clinical report 142 is generated. The set of tools 205 can be identified, which are actionable for the patient and associated with the extracted and normalized recommendation elements.

At 340, the patient actionable clinical report 142 is displayed on a display device. The display can include icons 200 and/or links to the set of tools 205.

At 350, at least one of the set of tools 205 is executed. The set of tools 205 can comprise at least one of a patient scheduler for a medical examination, test, or completing a questionnaire. Related to the action, the set of tools can include a healthcare provider locator, background information link, chat session with a healthcare practitioner, phone or voice contact with a healthcare practitioner. Each of the tools can include submenus, which indicate selections corresponding to the tools, such as different locator options, different search terms, different types of healthcare practitioners, different scheduling options, and the like.

The above may be implemented by way of computer readable instructions, encoded or embedded on a computer readable storage medium, which, when executed by a computer processor(s), cause the processor(s) to carry out the described acts. Additionally or alternatively, at least one of the computer readable instructions is carried by a signal, carrier wave or other transitory medium.

The invention has been described with reference to the preferred embodiments. Modifications and alterations may occur to others upon reading and understanding the preceding detailed description. It is intended that the invention is constructed as including all such modifications and alterations insofar as they come within the scope of the appended claims or the equivalents thereof. The word “comprising” does not exclude other elements or steps, and the indefinite article “a” or “an” does not exclude a plurality.

1. A system, comprising:
a parser engine configured to detect a recommendation in a received clinical report for a patient;
a context inferring engine configured to extract and normalize recommendation elements in the detected recommendation; and
an action selection engine configured to generate a patient actionable clinical report based on the received clinical report and the extracted and normalized recommendation elements.

2. The system according to claim 1, wherein the recommendation elements include an action and a timeframe.

3. The system according to claim 1, wherein the action selection engine is further configured to:
select a set of tools corresponding to the action that are patient actionable.

4. The system according to claim 1, wherein the generated patient actionable clinical report includes an indicator that identifies the detected recommendation.

5. The system according to claim 4, wherein the identifier includes a hyperlink to the selected set of tools.

6. The system according to claim 1, further including:
a user interface configured to display the generated patient actionable clinical report on a display device.

7. The system according to claim 6, wherein the display of the displayed patient actionable clinical report further comprises the selected set of tools.

8. The system according to claim 2, wherein the action comprises at least one selected from a group comprised of an imaging examination, a laboratory test, and a questionnaire;
wherein one tool from the selected set of tools comprises a scheduling tool.

9. A method, comprising:
detecting a recommendation in a received clinical report for a patient;
extracting and normalizing recommendation elements in the detected recommendation; and
generating a patient actionable clinical report based on the received clinical report and the extracted and normalized recommendation elements.

10. The method according to claim 9, wherein the recommendation elements include an action and a timeframe.

11. The method according to claim 9, wherein generating the patient action clinical report further comprises:
selecting a set of tools corresponding to the action that are patient actionable.

12. The method according to claim 9, wherein the generated patient actionable clinical report includes an indicator that identifies the detected recommendation.

13. The method according to claim 9, further including:
displaying the generated patient actionable clinical report on a display device.

14. The method according to claim 9, wherein the display of the displayed patient actionable clinical report further comprises the selected set of tools.

15. The method according to claim 11, wherein the action comprises at least one selected from a group comprised of an imaging examination, a laboratory test, and a questionnaire;
wherein one tool from the selected set of tools comprises a scheduling tool.

16. A non-transitory computer-readable storage medium carrying instructions which controls one or more processors to:
detect a recommendation in a received clinical report for a patient;
extract and normalize recommendation elements in the detected recommendation; and
generate a patient actionable clinical report based on the received clinical report and the extracted and normalized recommendation elements.

17. The non-transitory computer-readable storage medium according to claim 16, wherein the recommendation elements include an action and a timeframe.

18. The non-transitory computer-readable storage medium according to claim 16, wherein the one or more processors are further controlled to:
select a set of tools corresponding to the action that are patient actionable.

19. The non-transitory computer-readable storage medium according to claim 16, wherein the one or more processors are further controlled to:
display the generated patient actionable clinical report on a display device.

20. The non-transitory computer-readable storage medium according to claim 16, wherein the action comprises at least one selected from a group comprised of an imaging examination, a laboratory test, and a questionnaire;
wherein one tool from the selected set of tools comprises a scheduling tool.
